(19) United States
(12) Patent Application Publication
(10) Pub. No.: US 2025/0342967 A1
(43) Pub. Date: Nov. 6, 2025

(54) SYSTEMS AND METHODS FOR EXTRACTING CLINICAL PHENOTYPES FOR ALZHEIMER DISEASE DEMENTIA FROM UNSTRUCTURED CLINICAL RECORDS USING NATURAL LANGUAGE PROCESSING

(71) Applicant: Washington University, St. Louis, MO (US)

(72) Inventors: Inez OH, St. Louis, MO (US); Nupur GHOSHAL, St. Louis, MO (US); Aditi GUPTA, St. Louis, MO (US); Albert LAI, St. Louis, MO (US); Philip PAYNE, Frontenac, MO (US); Suzanne SCHINDLER, St. Louis, MO (US)

(21) Appl. No.: 18/855,529

(22) PCT Filed: Apr. 13, 2023
(86) PCT No.: PCT/US2023/018540

§ 371 Date: Oct. 9, 2024

Related U.S. Application Data
(60) Provisional application No. 63/331,120, filed on Apr. 14, 2022.

(51) Int. Cl. G16H 50/30; G06F 40/205; G16H 10/60

(52) U.S. Cl. CPC G16H 50/30; G06F 40/205; G16H 10/60

(57) ABSTRACT

An analytics computing device is provided. The analytics computing device includes a processor in communication with a database. The database configured to store electronic health record (EHR) data including structured EHR data and unstructured EHR data for a patient. The processor is configured to retrieve the EHR data from the database. The processor is further configured to parse, using a natural language processing model, the unstructured EHR data to retrieve one or more indicator phrases, the one or more indicator phrases correlated to an Alzheimer's disease (AD) diagnosis. The processor is further configured to identify, using a predictive model, the patient as being at risk for AD based on the retrieved indicator phrases and on the structured EHR data.

### FIELD OF USE

The present disclosure relates to clinical data analytics and, more particularly, to systems and methods for extracting clinical phenotypes (e.g., observable traits or indicators) for Alzheimer Disease (AD) dementia from clinical records using natural language processing (NPL).

### BACKGROUND

Computers may be used by physicians and researcher to analyze clinical data for making predictions about patient outcomes. For example, a major area of research in the AD domain is how to identify individuals who will develop AD, which AD patients will progress to severe stages of the disease, and how quickly the progression will occur. Hence, there has been much impetus to develop clinical predictive models for AD dementia to address these questions. However, existing systems generally utilize only structured Electronic Health Record (EHR) data or curated research registries. EHR data collected over the course of routine patient care is a valuable resource for predicting the clinical trajectory of AD dementia.

However, much of the critical information relevant to AD dementia resides in relatively inaccessible unstructured clinical notes or records within the EHR. Such data may include, for example, including medical comorbidities, biomarkers, neurobehavioral test scores, behavioral indicators of cognitive decline, family history, and neuroimaging findings, which are important for accurately analyzing a patient's risk of developing AD. A computing device capable of extracting this unstructured data for use within a predictive model for AD is therefore desirable.

### BRIEF SUMMARY

In one aspect, an analytics computing device is provided. The analytics computing device includes a processor in communication with a database. The database configured to store electronic health record (EHR) data including structured EHR data and unstructured EHR data for a patient. The processor is configured to retrieve the EHR data from the database. The processor is further configured to parse, using a natural language processing model, the unstructured EHR data to retrieve one or more indicator phrases, the one or more indicator phrases correlated to an Alzheimer's disease (AD) diagnosis. The processor is further configured to identify, using a predictive model, the patient as being at risk for AD based on the retrieved indicator phrases and on the structured EHR data.

In another aspect, a computing-implemented method for analyzing a likelihood of a patient developing AD based on EHR data is provided. The computer-implemented method is performed by an analytics computing device including a processor in communication with a database. The database is configured to store the EHR data including structured EHR data and unstructured EHR data. The computer-implemented method includes retrieving, by the processor, the EHR data from the database. The computer-implemented method further includes parsing, by the processor, using a natural language processing model, the unstructured EHR data to retrieve one or more indicator phrases, the one or more indicator phrases correlated to an AD diagnosis. The computer-implemented method further includes identifying, by the processor, using a predictive model, the patient as being at risk for AD based on the retrieved indicator phrases and on the structured EHR data.

In another aspect, at least one non-transitory computer-readable media having computer-executable instructions embodied thereon is provided. When executed by an analytics computing device including a processor in communication with a database, the database configured to store EHR data including structured EHR data and unstructured EHR data for a patient, the computer-executable instructions cause the processor to retrieve the EHR data from the database. The computer-executable instructions further cause the processor to parse, using a natural language processing model, the unstructured EHR data to retrieve one or more indicator phrases, the one or more indicator phrases correlated to an Alzheimer's disease (AD) diagnosis. The computer-executable instructions further cause the processor to identify, using a predictive model, the patient as being at risk for AD based on the retrieved indicator phrases and on the structured EHR data.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and are instrumentalities shown, wherein:

FIG. 1 depicts an exemplary analytics system in accordance with an exemplary embodiment of the present disclosure.

FIG. 2 depicts an exemplary client computing device that may be used with the analytics system illustrated in FIG. 1.

FIG. 3 depicts an exemplary server system that may be used with the analytics system illustrated in FIG. 1.

FIG. 4 illustrates an exemplary computer-implemented method for analyzing a likelihood of a patient developing AD based on EHR that may be performed using the analytics system illustrated in FIG. 1.

The Figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION OF THE DRAWINGS

The present embodiments may relate to systems and methods for analyzing a likelihood of a patient developing AD based on EHR data that includes clinical notes and/or records. The EHR data may include structured EHR data and unstructured EHR data (e.g., clinical notes in a text format). The systems and methods may include retrieving the EHR data from a database. The database may include EHR data corresponding to, for example, many patients, and the retrieved EHR data may correspond to a patient who is to be assessed for a likelihood of developing AD. In the example embodiment, the unstructured EHR data may be formatted as plain text, which a requirement of certain NLP platforms (e.g., Linguamatics I2E). Alternatively, in some embodiments, the unstructured EHR data may be stored in other formats. The plain text notes may stored together with metadata (e.g., a patient ID, date of note creation, author, etc.) in, for example, a CSV file format.

The systems and methods may further include parsing, using a natural language processing model, the unstructured EHR data to retrieve one or more indicator phrases (e.g., from list of indicator words and/or phrases defined by a subject matter expert), wherein the one or more indicator phrases (e.g., clinical phenotypes or other features/characteristics/traits correlated with developing AD) correlated to AD diagnosis. Because the unstructured EHR data includes data useful for determining whether the patient is likely to develop AD, parsing the unstructured EHR data to identify and capture the indicator phrases improves the ability of the system to make predictions corresponding to the patient's likelihood of developing AD, because both structured and unstructured EHR data may be used to generate the prediction. In some embodiments, the extracted clinical phenotypes of interest may be stored in a tabular format (e.g., a CSV file). In such embodiments, the table may also contain columns for the metadata (e.g., patient/encounter IDs, dates, etc.) that serves to contextualize the note. Such metadata may be used for linking the data extracted from the notes to correlative, structured data.

The systems and methods may further identify, using a predictive model (e.g., a machine learning (ML) or artificial intelligence (AI) model), the patient as being at risk for developing AD based on the retrieved indicator phrases and on the structured EHR data. In some embodiments, the predictive model is built by the system using EHR data as training data.

In an example embodiment, the process described herein may be performed by an analytics computing device. The analytics computing device may include a processor in communication with a database or other memory. The database is configured to store electronic health record (EHR) data for one or more patients, and enable retrieval of said data. This EHR data may include information that may be used to, for example, predict whether a patent will develop AD, such as information regarding the applicability of various AD risk factors to the patient. The EHR data may include structured EHR data and unstructured EHR data. The structured EHR data may include data that has been stored in a predefined data structures, and may include information such as demographics, diagnoses, laboratory results, medications, procedures, or vital signs. Unstructured EHR data may include data (e.g., text data) that represents unstructured narratives, such as clinical notes taken by physicians, and metadata associated with such notes. This data may include, for example, clinical notes relating to a patient's cognitive concerns, changes in behavior, personal or family medical history, or ability to perform daily activities.

In the example embodiment, the analytics computing device may be configured to retrieve the EHR data from the database. For example, a physician may wish to determine a susceptibility to AD (e.g., likelihood of developing AD, rate of progression of the AD, etc.) for a certain patient, in which case the analytics computing device may retrieve structured EHR data and unstructured EHR data associated with the patient in the database. As described in further detail below, the retrieved EHR data may be used by the analytics computing device to determine, for example, whether the patient is likely (e.g., has a chance above a threshold chance) of developing AD.

In the example embodiment, the analytics computing device may be further configured to parse, using a natural language processing model (e.g., text mining), the unstructured EHR data to retrieve one or more indicator phrases. These indicator phrases may be stored in a list of indicator phrases in the database, and may be determined based on other machine learning techniques. The one or more indicator phrases may be correlated to AD diagnosis. For example, the indicator phrases may related to clinical phenotypes correlated with an increased chance of developing AD, such as indicators of a family history of AD, medical indicators (e.g., cognitive performance test results, lab test results, and/or other indicators) correlated with AD, or environmental risk factors correlated with AD. In some embodiments, to parse the unstructured EHR data for the one or more indicator phrases, analytics computing device may be configured to parse the unstructured EHR data using one or more ontologies that associate the indicator phrases with the clinical phenotypes at a contextual level. For example, the analytics computing device may search for the word “misplace,” and also search for spelling errors and different word morphologies (e.g., misplacing, misplaced). The analytics computing device may exclude results where a negation (e.g. “does not”, “denies”) appears right before the word “misplace.” The use of these ontologies allows the analytics computing device to retrieve information at a conceptual level without needing prior exhaustive knowledge of all synonyms and relationships subsumed under a concept. In certain embodiments, these ontologies may be defined according to a predefined NLP standard (e.g., Linguamatics I2E).

To further illustrate the use of ontologies, a query for family history of dementia may be performed by the analytics computing device when analyzing an unstructured text document (a “note”) as follows. Certain terms (“ontology terms”) may be associated with certain categories such as, for example, “dementia” (e.g., terms relating to dementia and/or AD), “genetic relations” (e.g., terms describing genetic relationships of the patient to different persons), “disease” (e.g., terms describing diseases), and/or “symptoms” (e.g., terms describing disease symptoms). A query may be performed, for example, for a phrase containing a “dementia” ontology term and a “genetic relations” ontology term occurring in any order within a set number (e.g., five) words of each other, with no other “disease” or “symptoms” ontology term within the set number of words. The analytics computing device may identify a section in the note pertaining to family history (e.g., by search for a “Family hx” phrase marking the start of the family history section). The analytics computing device may determine if the phrase returned by the query occurs after the “Family hx” phrase, and if it does, determine that the patient has a family history of dementia and identify the returned ontology term associated with the “genetic relations” category as the relative of the patient who has and/or had dementia and/or AD. When performing the query, the analytics computing device may account for negations, for example, by excluding results containing negative phrases such as “denied Alzheimer disease.”

In the example embodiment, the analytics computing device may be further configured to identify, using a predictive model, the patient as being at risk for AD based on the retrieved indicator phrases and on the structured EHR data. The analytics computing device may determine that the patient is at risk, for example, based on a comparison of a score or metric to a threshold. In certain embodiments, the analytics computing device may generate further predictions for the patient based on the predictive model, such as a rate at which AD may progress for the patient and/or an age at which the patient is likely to develop symptoms of AD.

In some embodiments, the predictive model is a machine learning (ML) model defining a relationship between the various inputs (e.g., structured EHR data and the clinical phenotypes extracted from unstructured EHR data) with AD outcomes (e.g., a likelihood of the patient developing AD and/or a rate at which AD is likely to develop for the patient). The ML model may be trained using EHR data associated with, for example, a large number of patients, to correlate various risk factors that may be extracted from the EHR data with clinical outcomes relating to AD. For example, in some embodiments, the analytics computing device may train the ML model based on EHR data stored in the database.

At least one of the technical problems addressed by this system may include: (i) inability of a computing device to extract clinical phenotypes related to AD diagnosis from unstructured EHR data; (ii) inability of a computing device to develop a predictive model for AD based on unstructured EHR data; and/or (iii) inability of a computing device to identify patients as at risk for AD based on unstructured EHR data.

A technical effect of the systems and processes described herein may be achieved by performing at least one of the following steps: (i) retrieving EHR data including structured EHR data and unstructured EHR data from a database; (ii) parsing, using a natural language processing model, the unstructured EHR data to retrieve one or more indicator phrases, the one or more indicator phrases correlated to AD diagnosis; and/or (iii) identifying, using a predictive model, the patient as being at risk for AD based on the retrieved indicator phrases and on the structured EHR data.

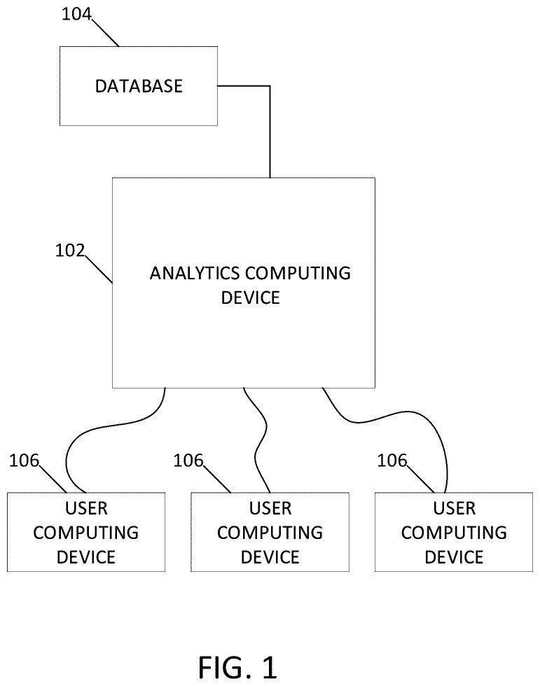

FIG. 1 depicts an exemplary analytics system 100. Analytics system 100 may include an analytics computing device 102 in communication with a database 104. Analytics computing device 102 may further be in communication with one or more user devices 106. User devices 106 may be, for example, personal computers, tablets, mobile phone device, or other computing devices capable of communicating with analytics computing device 102. In some embodiments, analytics computing device 102 is configured to cause the one or more user devices to display a user interface though which users (e.g., physicians) may interact with server computing device. For example, a physician may request that analytics computing device 102 analyze a patent's records to determine whether the patient is likely to develop AD, and view the results of the analysis via the user interface.

Database 104 is configured to store EHR data to retrieve one or more patients. This EHR data may include information that may be used to, for example, predict whether a patent will develop AD, such as information regarding the applicability of various AD risk factors to the patient. The EHR data may include structured EHR data and unstructured EHR data. The structured EHR data includes data that has been stored in a predefined data structures, and may include information such as demographics, diagnoses, laboratory results, medications, procedures, or vital signs. Unstructured EHR data includes data (e.g., text data) that represents unstructured narratives, such as clinical notes taken by physicians. This data may include, for example, clinical notes relating to a patient's cognitive concerns, changes in behavior, personal or family medical history, or ability to perform daily activities.

In the example embodiment, analytics computing device 102 may be configured to retrieve the EHR data from the database. For example, a physician may wish to determine a susceptibility to AD (e.g., likelihood of developing AD, rate of progression of the AD) for a certain patient, in which case analytics computing device 102 may retrieve structured EHR data and unstructured EHR data associated with the patient in the database. As described in further detail below, the retrieved EHR data may be used by analytics computing device 102 to determine, for example, whether the patient is likely (e.g., has a chance above a threshold chance) of developing AD.

In the example embodiment, analytics computing device 102 may be further configured to parse, using a natural language processing model, the unstructured EHR data to retrieve one or more indicator phrases. The one or more indicator phrases may be correlated to Alzheimer's disease (AD) diagnosis. For example, the indicator phrases may related to clinical phenotypes correlated with an increased chance of developing AD, such as indicators of a family history of AD, medical indicators (e.g., cognitive performance test results, lab test results, and/or other indicators) correlated with AD, or environmental risk factors correlated with AD. In some embodiments, to parse the unstructured EHR data for the one or more indicator phrases, analytics computing device 102 may be configured to parse the unstructured EHR data using one or more ontologies that associate the indicator phrases with the clinical phenotypes at a contextual level. For example, analytics computing device 102 may search for the word “misplace,” and also search for spelling errors and different word morphologies (e.g. misplacing, misplaced). Analytics computing device 102 may exclude results where a negation (e.g. “does not”, “denies”) appears right before the word “misplace.” The use of these ontologies allows analytics computing device 102 to retrieve information at a conceptual level without needing prior exhaustive knowledge of all synonyms and relationships subsumed under a concept. In certain embodiments, these ontologies may be defined according to a predefined NLP standard (e.g., Linguamatics I2E).

To further illustrate the use of ontologies, a query for family history of dementia may be performed by analytics computing device 102 when analyzing an unstructured text document (a “note”) as follows. Certain terms (“ontology terms”) may be associated with certain categories such as, for example, “dementia” (e.g., terms relating to dementia and/or AD), “genetic relations” (e.g., terms describing genetic relationships of the patient to different persons), “disease” (e.g., terms describing diseases), and/or “symptoms” (e.g., terms describing disease symptoms). A query may be performed, for example, for a phrase containing a “dementia” ontology term and a “genetic relations” ontology term occurring in any order within a set number (e.g., five) words of each other, with no other “disease” or “symptoms” ontology term within the set number of words. Analytics computing device 102 may identify a section in the note pertaining to family history (e.g., by search for a “Family hx” phrase marking the start of the family history section). Analytics computing device 102 may determine if the phrase returned by the query occurs after the “Family hx” phrase, and if it does, determine that the patient has a family history of dementia and identify the returned ontology term associated with the “genetic relations” category as the relative of the patient who has and/or had dementia and/or AD. When performing the query, analytics computing device 102 may account for negations, for example, by excluding results containing negative phrases such as “denied Alzheimer disease.”

In the example embodiment, analytics computing device 102 may be further configured to identify, using a predictive model, the patient as being at risk for AD based on the retrieved indicator phrases and on the structured EHR data. Analytics computing device 102 may determine that the patient is at risk, for example, based on a comparison of a score or metric to a threshold. In certain embodiments, analytics computing device 102 may generate further predictions for the patient based on the predictive model, such as a rate at which AD may progress for the patient and/or an age at which the patient is likely to develop symptoms of AD.

In some embodiments, the predictive model is a machine learning (ML) model defining a relationship between the various inputs (e.g., structured EHR data and the clinical phenotypes extracted from unstructured EHR data) with AD outcomes (e.g., a likelihood of the patient developing AD and/or a rate at which AD is likely to develop for the patient). The ML model may be trained using EHR data associated with, for example, a large number of patients, to correlate various risk factors that may be extracted from the EHR data with clinical outcomes relating to AD. For example, in some embodiments, analytics computing device 102 may train the ML model based on EHR data stored in the database.

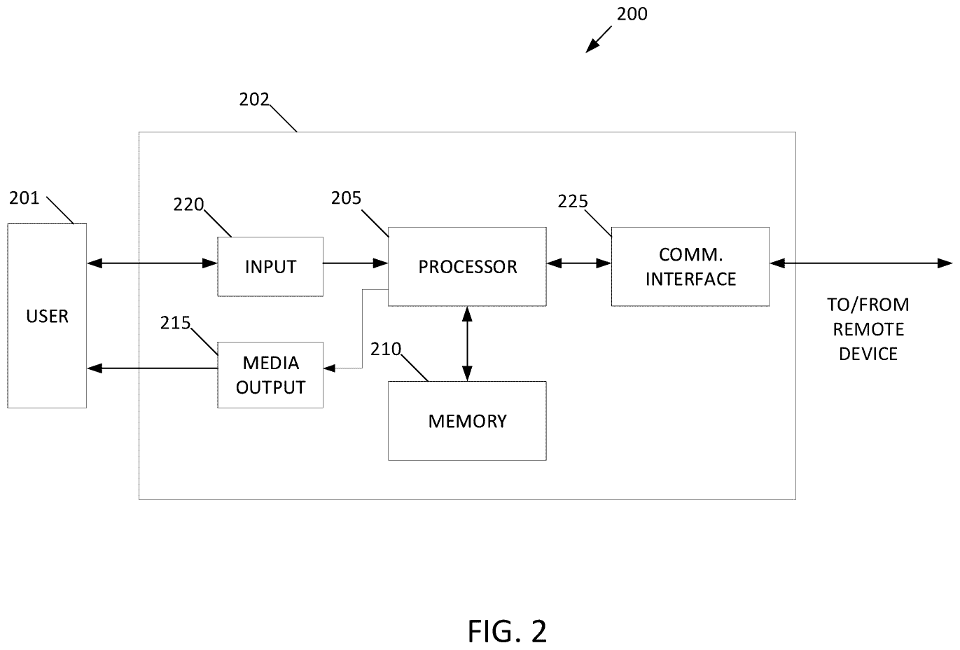

FIG. 2 depicts an exemplary client computing device 202. Client computing device 202 may be, for example, at least one of user devices 106 (shown in FIG. 1).

Client computing device 202 may include a processor 205 for executing instructions. In some embodiments, executable instructions may be stored in a memory area 210. Processor 205 may include one or more processing units (e.g., in a multi-core configuration). Memory area 210 may be any device allowing information such as executable instructions and/or other data to be stored and retrieved. Memory area 210 may include one or more computer readable media.

In exemplary embodiments, client computing device 202 may also include at least one media output component 215 for presenting information to a user 201. Media output component 215 may be any component capable of conveying information to user 201. In some embodiments, media output component 215 may include an output adapter such as a video adapter and/or an audio adapter. An output adapter may be operatively coupled to processor 205 and operatively couplable to an output device such as a display device (e.g., a liquid crystal display (LCD), light emitting diode (LED) display, organic light emitting diode (OLED) display, cathode ray tube (CRT) display, “electronic ink” display, or a projected display) or an audio output device (e.g., a speaker or headphones).

Client computing device 202 may also include an input device 220 for receiving input from user 201. Input device 220 may include, for example, a keyboard, a pointing device, a mouse, a stylus, a touch sensitive panel (e.g., a touch pad or a touch screen), a gyroscope, an accelerometer, a position detector, or an audio input device. A single component such as a touch screen may function as both an output device of media output component 215 and input device 220.

Client computing device 202 may also include a communication interface 225, which can be communicatively coupled to a remote device such as analytics computing device 102 (shown in FIG. 1). Communication interface 225 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 210 may be, for example, computer readable instructions for providing a user interface to user 201 via media output component 215 and, optionally, receiving and processing input from input device 220. A user interface may include, among other possibilities, a web browser and client application. Web browsers may enable users, such as user 201, to display and interact with media and other information typically embedded on a web page or a website. A client application may allow user 201 to interact with a server application from analytics computing device 102 (shown in FIG. 1).

Memory area 210 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

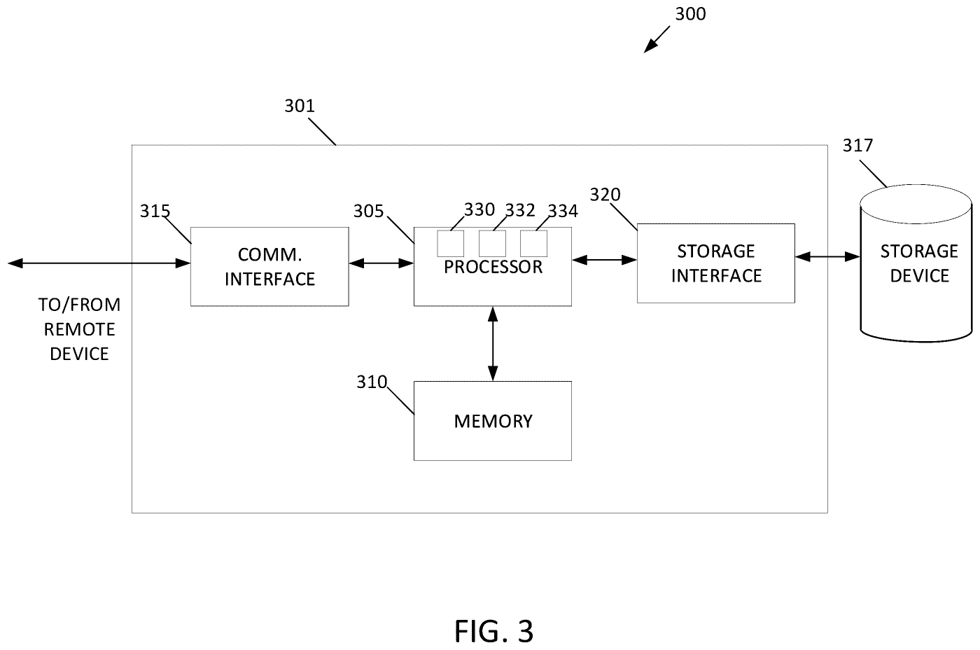

FIG. 3 depicts an exemplary server system that may be used with the analytics system illustrated in FIG. 1. Server system 301 may be, for example, analytics computing device 102 (shown in FIG. 1).

In exemplary embodiments, server system 301 may include a processor 305 for executing instructions. Instructions may be stored in a memory area 310. Processor 305 may include one or more processing units (e.g., in a multi-core configuration) for executing instructions. The instructions may be executed within a variety of different operating systems on server system 301, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C #, C++, Java, or other suitable programming languages, etc.).

In exemplary embodiments, processor 305 may include and/or be communicatively coupled to one or more modules for implementing the systems and methods described herein. Processor 305 may include a data management module 330 configured for retrieve the EHR data from a database (e.g., database 104). Processor 305 may further include a language processing module 332 configured for parsing, using a natural language processing model, the unstructured EHR data to retrieve one or more indicator phrases to AD diagnosis. Processor 305 may further includes a prediction module 334 configured for identifying, using a predictive model, a patient as being at risk for AD based on the retrieved indicator phrases and on structured EHR data.

Processor 305 may be operatively coupled to a communication interface 315 such that server system 301 is capable of communicating with user devices 106 (shown in FIG. 1), or another server system 301. For example, communication interface 315 may receive requests from user device 106 via the Internet.

Processor 305 may also be operatively coupled to a storage device 317, such as database 104 (shown in FIG. 1). Storage device 317 may be any computer-operated hardware suitable for storing and/or retrieving data. In some embodiments, storage device 317 may be integrated in server system 301. For example, server system 301 may include one or more hard disk drives as storage device 317.

In other embodiments, storage device 317 may be external to server system 301 and may be accessed by a plurality of server systems 301. For example, storage device 317 may include multiple storage units such as hard disks or solid state disks in a redundant array of inexpensive disks (RAID) configuration. Storage device 317 may include a storage area network (SAN) and/or a network attached storage (NAS) system.

In some embodiments, processor 305 may be operatively coupled to storage device 317 via a storage interface 320. Storage interface 320 may be any component capable of providing processor 305 with access to storage device 317. Storage interface 320 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 305 with access to storage device 317.

Memory area 310 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

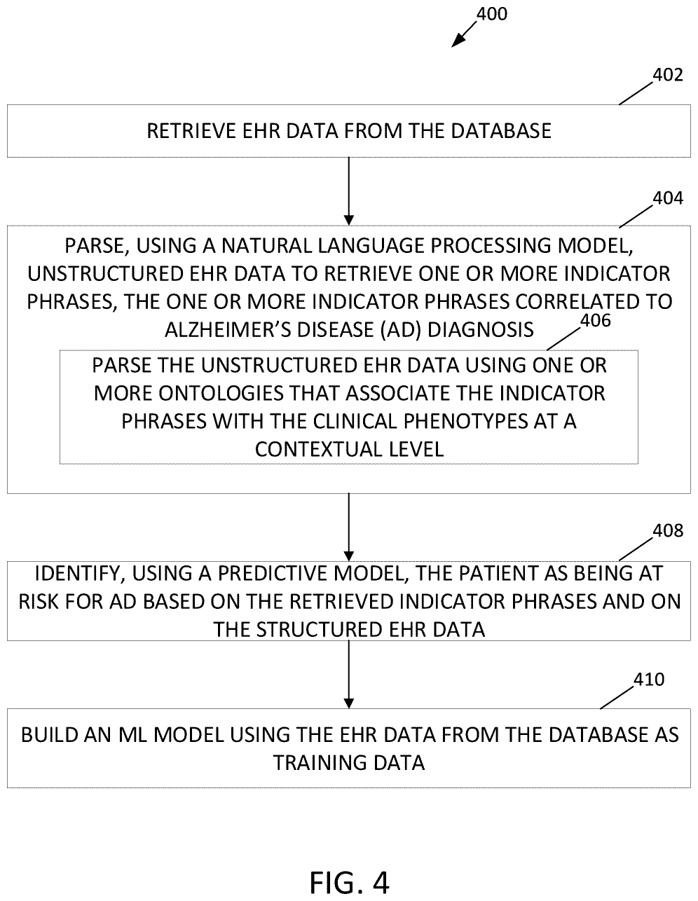

FIG. 4 depicts an example computer-implemented method 400 for analyzing a likelihood of a patient developing AD based on EHR data. Computer-implemented method 400 may be performed, for example, by analytics computing device 102 (shown in FIG. 1). The EHR data may include structured EHR data and unstructured EHR data for a patient, and may be stored in a database such as database 104 (shown in FIG. 1).

Computer-implemented method 400 may include retrieving 402 the EHR data from the database. In some embodiments, retrieving 402 the EHR data may be performed by analytics computing device 102 by executing data management module 330 (shown in FIG. 3).

Computer-implemented method 400 may further include parsing 404, using a natural language processing model, the unstructured EHR data to retrieve one or more indicator phrases. The one or more indicator phrases may be correlated to AD diagnosis. In certain embodiments, the indicator phrases are associated with clinical phenotypes. In some such embodiments, parsing 404 unstructured EHR data for the one or more indicator phrases includes parsing 406 the unstructured EHR data using one or more ontologies that associate the indicator phrases with the clinical phenotypes at a contextual level. In some embodiments, parsing 404 the unstructured EHR data may be performed by analytics computing device 102 by executing language processing module 332 (shown in FIG. 3).

Computer-implemented method 400 may further include identifying 408, using a predictive model, the patient as being at risk for AD based on the retrieved indicator phrases and on the structured EHR data. In certain embodiments, the predictive model is a ML model. In some such embodiments, computer-implemented method 400 further includes building 410 the ML model using the EHR data from the database as training data. In some embodiments, identifying 408 the patient as being at risk for AD and/or building 410 the ML model may be performed by analytics computing device 102 by executing prediction module 334 (shown in FIG. 3).

The computer-implemented methods discussed herein may include additional, less, or alternate actions, including those discussed elsewhere herein. The methods may be implemented via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

A processor or a processing element may be trained using supervised or unsupervised machine learning, and the machine learning program may employ a neural network, which may be a convolutional neural network, a deep learning neural network, or a combined learning module or program that learns in two or more fields or areas of interest. Machine learning may involve identifying and recognizing patterns in existing data in order to facilitate making predictions for subsequent data. Models may be created based on example inputs in order to make valid and reliable predictions for novel inputs.

Additionally or alternatively, the machine learning programs may be trained by inputting sample data sets or certain data into the programs, such as images, object statistics and information, historical estimates, and/or actual repair costs. The machine learning programs may utilize deep learning algorithms that may be primarily focused on pattern recognition, and may be trained after processing multiple examples. The machine learning programs may include Bayesian program learning (BPL), reinforced learning techniques, voice recognition and synthesis, image or object recognition, optical character recognition, and/or natural language processing-either individually or in combination. The machine learning programs may also include natural language processing, semantic analysis, automatic reasoning, and/or other types of machine learning or artificial intelligence.

In supervised machine learning, a processing element may be provided with example inputs and their associated outputs, and may seek to discover a general rule that maps inputs to outputs, so that when subsequent novel inputs are provided the processing element may, based on the discovered rule, accurately predict the correct output. In unsupervised machine learning, the processing element may be required to find its own structure in unlabeled example inputs.

As described above, the systems and methods described herein may use machine learning, for example, for pattern recognition. That is, machine learning algorithms may be used by the analytics computing device to attempt to identify patterns within EHR data. Further, machine learning algorithms may be used by the analytics computing device to predict a patient's likelihood of developing AD based on the patterns. Accordingly, the systems and methods described herein may use machine learning algorithms for both pattern recognition and predictive modeling.

As will be appreciated based on the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In one embodiment, a computer program is provided, and the program is embodied on a computer readable medium. In an example embodiment, the system is executed on a single computer system, without requiring a connection to a sever computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

As used herein, an element or step recited in the singular and preceded by the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

The patent claims at the end of this document are not intended to be construed under 35 U.S.C. § 112 (f) unless traditional means-plus-function language is expressly recited, such as “means for” or “step for” language being expressly recited in the claim(s).

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An analytics computing device comprising a processor in communication with a database, the database configured to store electronic health record (EHR) data including structured EHR data and unstructured EHR data for a patient, the processor configured to:
retrieve the EHR data from the database;
parse, using a natural language processing model, the unstructured EHR data to retrieve one or more indicator phrases, the one or more indicator phrases correlated to an Alzheimer's disease (AD) diagnosis; and
identify, using a predictive model, the patient as being at risk for AD based on the retrieved indicator phrases and on the structured EHR data.

2. The analytics computing device of claim 1, wherein the indicator phrases are associated with clinical phenotypes.

3. The analytics computing device of claim 2, wherein to parse the unstructured EHR data for the one or more indicator phrases, the processor is configured to parse the unstructured EHR data using one or more ontologies that associate the indicator phrases with the clinical phenotypes at a contextual level.

4. The analytics computing device of claim 1, wherein the predictive model is a machine learning (ML) model.

5. The analytics computing device of claim 4, wherein the processor is further configured to build the ML model using the EHR data from the database as training data.

6. The analytics computing device of claim 1 wherein the unstructured EHR data includes clinical notes.

7. The analytics computing device of claim 6, wherein the clinical notes include information relating to one or more of cognitive concerns, changes in behavior, personal or family medical history, or ability to perform daily activities.

8. The analytics computing device of claim 1, wherein the structured EHR data includes one or more of demographics data, diagnoses data, laboratory results, medications data, procedures performed data, or vital signs data.

9. A computing-implemented method for analyzing a likelihood of a patient developing Alzheimer's disease (AD) based on electronic health record (EHR) data, the computer-implemented method performed by an analytics computing device including a processor in communication with a database, the database configured to store the EHR data including structured EHR data and unstructured EHR data, the computer-implemented method comprising:
retrieving, by the processor, the EHR data from the database;
parsing, by the processor, using a natural language processing model, the unstructured EHR data to retrieve one or more indicator phrases, the one or more indicator phrases correlated to an AD diagnosis; and
identifying, by the processor, using a predictive model, the patient as being at risk for AD based on the retrieved indicator phrases and on the structured EHR data.

10. The computer-implemented method of claim 9, wherein the indicator phrases are associated with clinical phenotypes.

11. The computer-implemented method of claim 10, wherein parsing the unstructured EHR data for the one or more indicator phrases comprises parsing, by the processor, the unstructured EHR data using one or more ontologies that associate the indicator phrases with the clinical phenotypes at a contextual level.

12. The computer-implemented method of claim 9, wherein the predictive model is a machine learning (ML) model.

13. The computer-implemented method of claim 12, further comprising building, by the processor, the ML model using the EHR data from the database as training data.

14. The computer-implemented method of claim 9 wherein the unstructured EHR data includes clinical notes.

15. The computer-implemented method of claim 14, wherein the clinical notes include information relating to one or more of cognitive concerns, changes in behavior, personal or family medical history, or ability to perform daily activities.

16. The computer-implemented method of claim 9, wherein the structured EHR data includes one or more of demographics data, diagnoses data, laboratory results data, medications data, procedures performed data, or vital signs data.

17. At least one non-transitory computer-readable media having computer-executable instructions embodied thereon, wherein when executed by an analytics computing device including a processor in communication with a database, the database configured to store electronic health record (EHR) data including structured EHR data and unstructured EHR data for a patient, the computer-executable instructions cause the processor to:
retrieve the EHR data from the database;
parse, using a natural language processing model, the unstructured EHR data to retrieve one or more indicator phrases, the one or more indicator phrases correlated to an Alzheimer's disease (AD) diagnosis; and
identify, using a predictive model, the patient as being at risk for AD based on the retrieved indicator phrases and on the structured EHR data.

18. The at least one non-transitory computer-readable media of claim 17, wherein the indicator phrases are associated with clinical phenotypes.

19. The at least one non-transitory computer-readable media of claim 18, wherein to parse the unstructured EHR data for the one or more indicator phrases, the computer-executable instructions further cause the processor to parse the unstructured EHR data using one or more ontologies that associate the indicator phrases with the clinical phenotypes at a contextual level.

20. The at least one non-transitory computer-readable media of claim 17, wherein the predictive model is a machine learning (ML) model, and wherein the computer-executable instructions further cause the processor to build the ML model using the EHR data from the database as training data.
